(19) United States
(12) Patent Application Publication
(10) Pub. No.: US 2026/0114978 A1
(43) Pub. Date: Apr. 30, 2026

(54) SURGICAL MESH WITH VARIABLE ELASTICITY

(71) Applicant: Ariel HALEVY, Rishon Le Zion (IL)

(72) Inventor: Ariel HALEVY, Rishon Le Zion (IL)

(21) Appl. No.: 19/484,331

(22) PCT Filed: May 15, 2024
(86) PCT No.: PCT/IL2024/050471

§ 371 Date: Nov. 13, 2025

(30) Foreign Application Priority Data
May 17, 2023 (IL) 303009

(51) Int. Cl. A61F 2/00

(52) U.S. Cl. CPC A61F 2/0063; A61F 2002/0068; A61F 2250/0018

(57) ABSTRACT

A surgical mesh comprising a first region having a first elasticity and at least one second region having second elasticity, the surgical mesh connectable to tissue by means of at least one suture. For each suture linking a portion of the surgical mesh to tissue, the region with the first elasticity and the at least one region with the second elasticity are configured to move in situ with the tissue such that the portion of the surgical mesh connected to a suture moves no more than 1 mm in relation to the tissue connected to the same suture.

### FIELD OF THE INVENTION

The present invention generally pertains to a system and method for providing a surgical mesh with variable elasticity.

### BACKGROUND OF THE INVENTION

Surgical meshes products are fabric meshes, typically knitted fabric, although woven or non-woven fabrics are used, that hold together the edges of a hernia. Typically, they are pinned to the tissue around their edges with fixation devices to hold them in place and to prevent the vertical edges of fabric, especially knitted fabric, from rolling into a tube; it is well-known that the vertical edges of knitted fabrics, especially stockinette stich fabric (known as plain knit fabric in the UK) will roll into a tube near the vertical edges. Common types of fixation device are sutures, pins, screws or corkscrew-type spirals; typically each is passed through the surgical mesh product near its edge and into the tissue. Use of a fixation device reduces the probability of failure of the product from about 30% within the first five years to about 10% within the first 5 years.

However, when a person with an inserted surgical mesh product sneezes or jumps, the impulsive force applied to the surgical mesh product can cause it to detach from one or more of the fixation devices, thereby allowing that portion of the mesh to roll up and encouraging detachment of more of the product from the fixation devices, leading to failure of the surgical mesh product and possible re-opening of the hernia. It is well-known in the art that 30% of fixated surgical mesh products will have failed within 10 years after implantation.

It is therefore a long felt need to provide a mesh for a fixatable surgical mesh product that has a smaller probability of failure than the prior-art meshes for fixatable surgical mesh products.

### SUMMARY OF THE INVENTION

It is an object of the present invention to disclose a system for providing a surgical mesh with variable elasticity.

It is another object of the present invention to disclose a surgical mesh comprising:
a first region having a first elasticity; and
at least one second region having second elasticity;
wherein, for each suture linking a portion of the first region to tissue, said first elasticity and
said at least one second elasticity are configured to move in situ said portion of the first region no more than 1 mm in relation to said tissue.

It is another object of the present invention to disclose the surgical mesh as described above, wherein said at least one second region is entirely contained within said first region.

It is another object of the present invention to disclose the surgical mesh as described above, wherein said at least one second region is either symmetrically located within said first region or asymmetrically located within said first region.

It is another object of the present invention to disclose the surgical mesh as described above, wherein said asymmetrical location is provided by a member of a group consisting of a location of a center of said at least one second region is different from a location of a center of said first region, an axis of said at least one second region is rotated relative to an axis of said first region or any combination thereof.

It is another object of the present invention to disclose the surgical mesh as described above, wherein said at least one second region comprises a plurality of second regions.

It is another object of the present invention to disclose the surgical mesh as described above, wherein, for at least one pair from said plurality of second regions, said second regions have different elasticities.

It is another object of the present invention to disclose the surgical mesh as described above, wherein at least one of said plurality of second regions is entirely contained within at least one other of said plurality of second regions.

It is another object of the present invention to disclose the surgical mesh as described above, wherein said at least one of said plurality of second regions is either symmetrically located within another of said plurality of second regions or asymmetrically located within said another of said plurality of second regions.

It is another object of the present invention to disclose the surgical mesh as described above, wherein said asymmetrical location is provided by a member of a group consisting of a location of a center of said at least one second region is different from a location of a center of said first region, an axis of said at least one second region is rotated relative to an axis of said first region or any combination thereof.

It is another object of the present invention to disclose the surgical mesh as described above, wherein said first region differs from said at least one second region by a difference selected from a group consisting of filament type, filament material, filament diameter, filament shape, mesh type, pore diameter, thickness or any combination thereof.

It is another object of the present invention to disclose the surgical mesh as described above, wherein said filament type is selected from a group consisting of monofilament, multifilament or any combination thereof.

It is another object of the present invention to disclose the surgical mesh as described above, wherein said filament material is selected from a group consisting of metal, a composite, a polymer, a biodegradable biomaterial or any combination thereof.

It is another object of the present invention to disclose the surgical mesh as described above, wherein said mesh type is selected from a group consisting of woven, knitted, crocheted, non-woven or any combination thereof.

It is another object of the present invention to disclose the surgical mesh as described above, wherein a shape of said at least one second region is selected from a group consisting of elliptical, polygonal, oval, lobed or any combination thereof.

It is another object of the present invention to disclose the surgical mesh as described above, wherein said shape of said at least one second region is either the same as a shape of said at least one first region, or different from a shape of said at least one first region.

It is another object of the present invention to disclose the surgical mesh as described above, wherein a shape of one of said plurality of second regions is either the same as a shape of another of said plurality of second regions, or different from a shape of another of said plurality of second regions.

It is another object of the present invention to disclose a method of attaching a surgical mesh comprising:
providing a surgical mesh comprising:
a first region having a first elasticity; and
at least one second region having second elasticity;
placing separable edges of a hernia in apposition;
emplacing the surgical mesh such that external edges of the surgical mesh extend beyond all edges of the hernia;
suturing the surgical mesh to tissue;
wherein, for each suture linking a portion of the first region to tissue, said first elasticity and said at least one second elasticity are configured to move in situ said portion of the first region no more than 1 mm in relation to said tissue.

It is another object of the present invention to disclose the method as described above, additionally comprising a step of providing said at least one second region entirely contained within said first region.

It is another object of the present invention to disclose the method as described above, additionally comprising a step of providing said at least one second region symmetrically located within said first region or asymmetrically located within said first region.

It is another object of the present invention to disclose the method as described above, additionally comprising a step of providing said asymmetrical location by a member of a group consisting of a location of a center of said at least one second region is different from a location of a center of said first region, an axis of said at least one second region is rotated relative to an axis of said first region or any combination thereof.

It is another object of the present invention to disclose the method as described above, additionally comprising a step of said at least one second region comprising a plurality of second regions.

It is another object of the present invention to disclose the method as described above, additionally comprising a step of, for at least one pair from said plurality of second regions, providing said second regions having different elasticities.

It is another object of the present invention to disclose the method as described above, additionally comprising a step of providing at least one of said plurality of second regions entirely contained within at least one other of said plurality of second regions.

It is another object of the present invention to disclose the method as described above, additionally comprising a step of providing said at least one of said plurality of second regions either symmetrically located within another of said plurality of second regions or asymmetrically located within said another of said plurality of second regions.

It is another object of the present invention to disclose the method as described above, additionally comprising a step of providing said asymmetrical location by a member of a group consisting of a location of a center of said at least one second region is different from a location of a center of said first region, an axis of said at least one second region is rotated relative to an axis of said first region or any combination thereof.

It is another object of the present invention to disclose the method as described above, additionally comprising a step of providing said first region differing from said at least one second region by a difference selected from a group consisting of filament type, filament material, filament diameter, filament shape, mesh type, pore diameter, thickness or any combination thereof.

It is another object of the present invention to disclose the method as described above, additionally comprising a step of selecting said filament type from a group consisting of monofilament, multifilament or any combination thereof.

It is another object of the present invention to disclose the method as described above, additionally comprising a step of selecting said filament material from a group consisting of metal, a composite, a polymer, a biodegradable biomaterial or any combination thereof.

It is another object of the present invention to disclose the method as described above, additionally comprising a step of selecting said mesh type from a group consisting of woven, knitted, crocheted, non-woven or any combination thereof.

It is another object of the present invention to disclose the method as described above, additionally comprising a step of selecting a shape of said at least one second region from a group consisting of elliptical, polygonal, oval, lobed or any combination thereof.

It is another object of the present invention to disclose the method as described above, additionally comprising a step of providing said shape of said at least one second region either the same as a shape of said at least one first region, or different from a shape of said at least one first region.

It is another object of the present invention to disclose the method as described above, additionally comprising a step of providing a shape of one of said plurality of second regions either the same as a shape of another of said plurality of second regions, or different from a shape of another of said plurality of second regions.

### BRIEF DESCRIPTION OF THE FIGURES

In order to better understand the invention and its implementation in practice, a plurality of embodiments will now be described, by way of non-limiting example only, with reference to the accompanying drawings, wherein

FIG. 1 schematically illustrates a surgical mesh product of the prior art;

FIG. 2A-B schematically illustrates stretching of a surgical mesh product of the prior art and a surgical mesh product of the present invention;

FIGS. 3-10 schematically illustrate embodiments of surgical mesh products of the present invention having two regions with different elasticities;

FIG. 11 schematically illustrate embodiments of surgical mesh products of the present invention having three regions with different elasticities;

FIGS. 12-13 schematically illustrate embodiments of surgical mesh products of the present invention having two regions with different elasticities where the inner region is not symmetrically located within the edge region; and

FIG. 14A-E schematically illustrate embodiments of meshes.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for generating a surgical mesh with variable elasticity.

The term ‘surgical mesh’ hereinafter refers to a mesh fabric intended for insertion into a living body in order to help prevent a hernia from recurring.

The term ‘mesh’ hereinafter refers to the open pattern used for the construction of at least a portion of the surgical mesh. Non-limiting examples of a mesh comprise a knit pattern, a woven pattern, or a non-woven pattern (such as, but not limited to, a pressed felt with a large number of pores).

The term ‘surgical mesh product’ hereinafter refers to a surgical device made of a surgical mesh.

The term ‘about’ refers to a value in a range from 20% greater than to 20% smaller than a defined value.

The present invention is configured to reduce the probability of recurrence of a hernia by providing a surgical mesh with different elasticities in different portions of the surgical mesh, in order to prevent a surgical mesh product from detaching from its fixation devices. In surgical mesh products of the prior art, detachment typically occurs when the person sneezes or jumps, as this applies an impulsive force to the surgical mesh product. Such an impulsive force is likely to stretch the edges of the surgical mesh product more than it stretches its center, as the time it takes for the impulsive force to travel to the center of the surgical mesh is likely to be longer than the impulsive force itself.

The maximum intra-abdominal pressure generated in a healthy adult occurs when coughing or jumping and is estimated to be approximately 170 mmHg. Therefore, a surgical mesh product used to repair abdominal hernias must withstand at least 180 mmHg (20 kPa) before failing.

The tension placed on the abdominal wall can be calculated using Laplace's law relating the tension, pressure, thickness, and diameter of the abdominal wall. If a thin-walled cylinder model is used, the total tensile strength is independent of the thickness of the layer. In that case, the parameters become a physiological tensile strength of 16 N/cm and the pressure is 20 kPa (2 N/cm² as the maximum pressure to be experienced in the intra-abdominal wall), and a longitudinal diameter of the abdominal wall of 32 cm.

Studies of human abdominal walls have demonstrated that, at the maximum tensile strength of 16 N/cm, the abdominal wall in males has a natural mean distension of 23%±7% and 15%±5% when tissue is stretched in a vertical and a horizontal direction, respectively. In females, a distension of 32%±17% and 17%±5% in vertical and horizontal stretching has been observed.

Preferably, the edge region of a surgical mesh product is less elastic than the central region, as the lower elasticity (greater rigidity) of the edge region will conduce to retaining the surgical mesh product on its fixation devices, by having the edge region move less relative to its underlying or overlying tissue compared to the amount of movement of the central region relative to its underlying or overlying tissue, thereby reducing the amount the edge region moves relative to the fixation devices. The greater elasticity of the central region allows the central portion of the surgical mesh to stretch more than the edge region, thereby further reducing the amount the edge region moves relative to the fixation devices.

The different elasticity in the surgical mesh can be generated by means of a filament of a different material, by a filament of a different diameter, by a filament of a different shape, by a difference in weave, by a difference in knitting pattern, by a difference in crochet pattern, by a difference in pore size or any combination thereof.

A surgical mesh product should be characterized by inertness, resistance to infection, the ability to maintain adequate long-term tensile strength to prevent early recurrence, rapid incorporation into the host tissue, adequate flexibility to avoid fragmentation, non-carcinogenic response, an ability to maintain or restore the natural respiratory movements of the abdominal wall, or any combination thereof.

The surgical mesh can comprise weaving, a warp knit, a weft knit, knit stitches, purl stitches, a commercially-available knit (such as but not limited to a Bard® mesh), a proprietary knit, or any combination thereof. The type of mesh is not germane to the patent as long as it provides the desired elasticity in the various regions of the hernia mech and provides at least some of the desirable characteristics as disclosed above.

The mesh can comprise metal, a composite, a polymer, a biodegradable material, a biomaterial or any combination thereof.

The mesh of the edge region can be at an angle to an axis of the surgical mesh or the surgical mesh product, where the angle is in a range between 0 and 180°. At least two portions of the mesh of the edge region can be at angles differing from each other.

The mesh of a central region can be at an angle to an axis of the surgical mesh or the surgical mesh product, where the angle is in a range between 0 and 180°. At least two portions of the mesh of the central region can be at angles differing from each other.

The angle of a mesh of a central region can differ from an angle of the edge region.

The angle of a mesh of a central region can differ from an angle of another central region.

The material(s) used in a surgical mesh product of the present invention is not germane to the patent, as long as it comprises at least some of the desirable characteristics as disclosed above.

The pore size of the mesh(es) is not germane to the patent, as long as the resulting surgical mesh product comprises at least some of the desirable characteristics as disclosed above.

Mesh filaments can be monofilament, multifilament (braided), a patch (for non-limiting example, laminated ePTFE) or otherwise non-woven. The type of filament is not germane to the patent, as long as the resulting surgical mesh product comprises at least some of the desirable characteristics as disclosed above.

A surgical mesh product can be supplied as a ready-to-use device or can be cut to size and shape at the time of implantation.

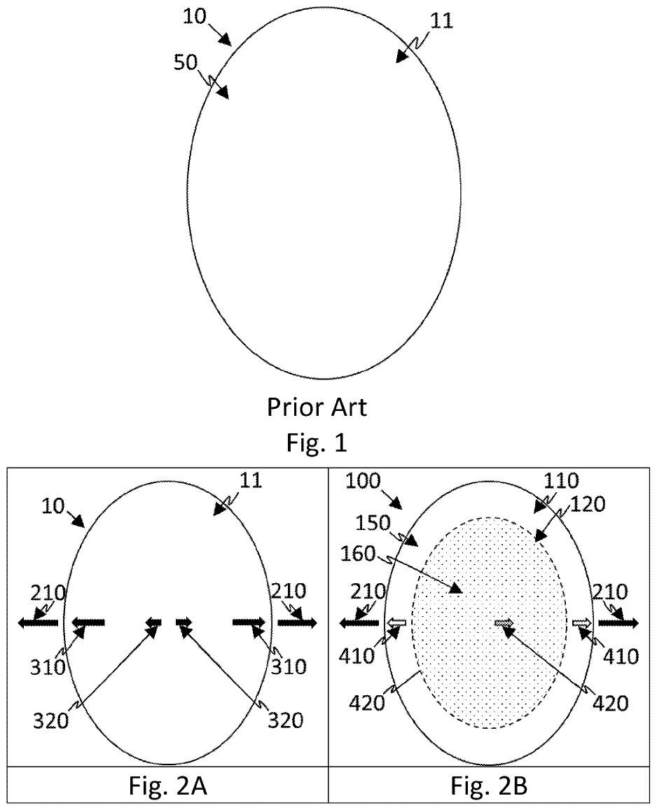

FIG. 1 schematically illustrates a surgical mesh product (10) of the prior art. It comprises a single region (50) having the same elasticity (11) throughout. It comprises a single material having a single type of mesh, and the filament(s) comprising the mesh do not change diameter or shape throughout.

FIG. 2A-B schematically illustrates an effect of having a more elastic region in a central portion of a surgical mesh. A surgical mesh product (10) of the prior art is schematically illustrated in FIG. 2A, while a surgical mesh product (100) of the present invention is schematically illustrated in FIG. 2B. The prior-art surgical mesh product (10, FIG. 2A) has a single elasticity (11) throughout, while the surgical mesh product of the present invention (100, FIG. 2B) has an edge region (150) with one elasticity (110) and a central region (160) with a second elasticity (120). Preferably, but not necessarily, the second, central elasticity (120) is greater than the first, edge elasticity (110).\

In FIGS. 2A-B, for simplicity, the same exemplary impulsive force (210) is applied to the surgical mesh product (10) of the prior art and the surgical mesh product of the present invention (100). For the surgical mesh product (10) of the prior art, the force (210) causes more stretching at the edges (50), schematically indicated by the white arrow (310), than near the center (60), schematically indicated by the grey arrow (320). In contrast, since the central region (160) of the surgical mesh product (100) of the present invention is more elastic than the edge region (150), the impulsive force (210) causes stretching of the edge region (150), schematically indicated by the white arrow (410), that is smaller than the stretching (310) near the edge (50) for the surgical mesh product (10) of the prior art. In addition, the stretching of the central region (160), schematically indicated by the grey arrow (420), is larger than the stretching (320) near the center (60) for the surgical mesh product (10) of the prior art.

Therefore, a surgical mesh product (100) of the present invention, by stretching the edge region less, reduces the probability that the surgical mesh product (100) will pull off its fixation devices.

The difference between the elasticity of the edge region (150) and the elasticity of the central region (160) can be adjusted so that the amount of stretching of the edge region (150) can be more than, the same as or less than the amount of stretching of the central region (160). By this means, in some embodiments, the amount of movement of the edge region can be “tuned” so that the edge region moves little, if at all, relative to the tissue underlying or overlying it, thereby reducing the probability that the fixation devices are pulled from the tissue.

By careful selection of a member of a group comprising the average elasticity of the surgical mesh product (100), the elasticity of the edge region (150), the elasticity of the at least one central region (160, 170), the shape of the surgical mesh product applied to the tissue, the size of the surgical mesh product applied to the tissue, the shape of the at least one central region (160, 170), the size of the at least one central region (160, 170), the type of fixation device, the spacing between the fixation devices, or any combination thereof, the probability of failure of the surgical mesh can be minimized, thereby significantly reducing the need for repeat operations to repair or replace the surgical mesh product, with a resulting reduction in patient discomfort, patient quality of life, and hospital costs.

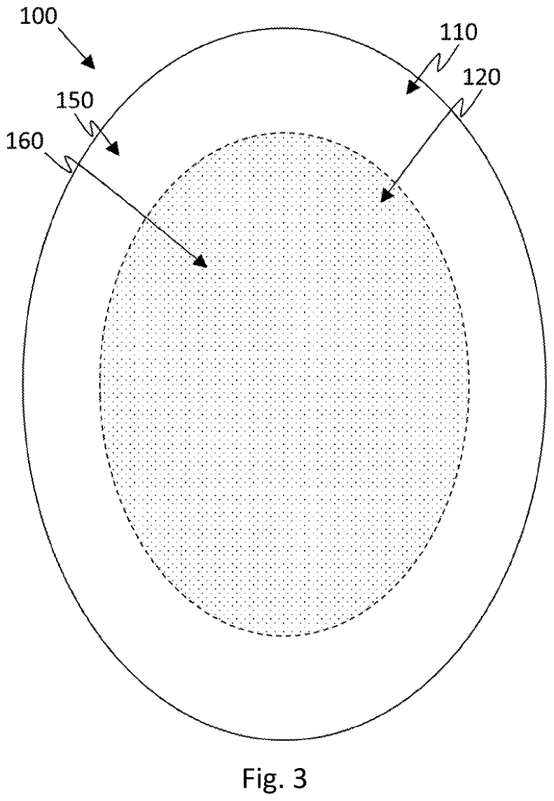

FIGS. 3-12 schematically illustrate, in a non-limiting manner, exemplary embodiments of surgical mesh products (100) with different sizes and shapes of central regions (160). For simplicity, the surgical mesh products (100) are shown as elliptical; in practice, they can be oval, elliptical, circular, polygonal, lobed or any combination thereof, with a polygonal product having a number of sides in a range from 2 to 20, and a lobed product having between three and 20 lobes.

FIG. 3 schematically illustrates a surgical mesh product (100) with an edge region (150) of one elasticity (110) and a central region (160) with a second elasticity (120), where the shape of the central region (160) is substantially the same as the shape of the surgical mesh product (100), so that the width of the edge region (150) is substantially the same everywhere.

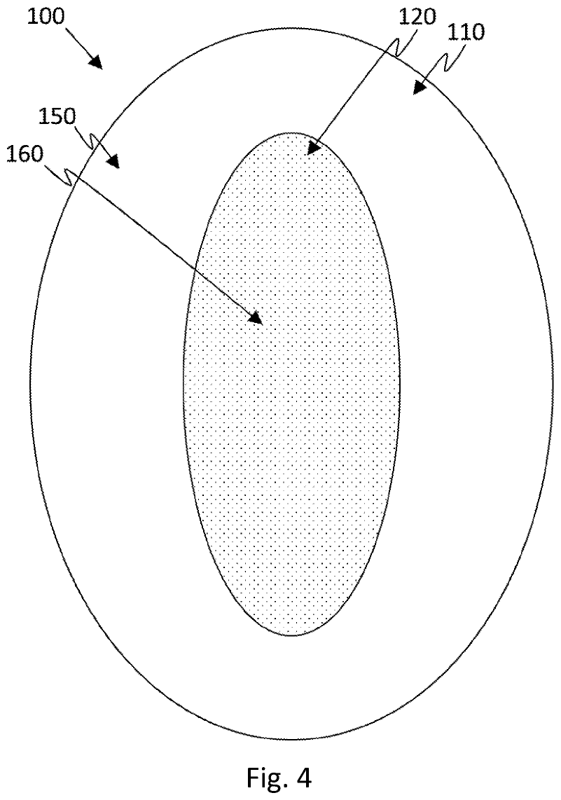

FIG. 4 schematically illustrates a surgical mesh product (100) with an edge region (150) of one elasticity (110) and a central region (160) with a second elasticity (120), where the basic shape of the central region (160) is the same as the basic shape of the surgical mesh product (100), in this case elliptical, with the centers of the ellipses at the same point and the axes of the ellipses parallel, but with the ellipses having different proportions. In FIG. 4, the edge region (150) is much wider at the sides of the surgical mesh product (100) than at the top and bottom, although the width of the edge region (150) is the same at the top and bottom and is the same on the left and right.

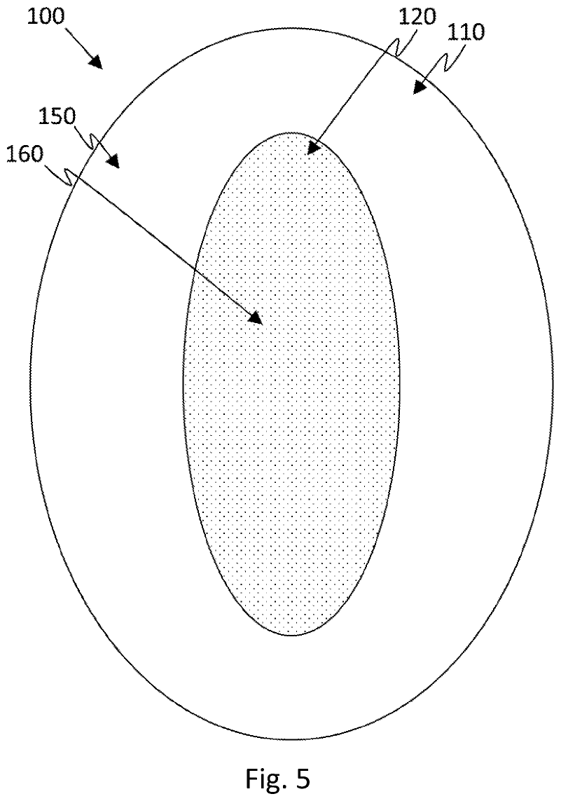

FIG. 5 schematically illustrates a surgical mesh product (100) with an edge region (150) of one elasticity (110) and a central region (160) with a second elasticity (120), where the basic shape of the central region (160) is the same as the basic shape of the surgical mesh product (100), in this case elliptical, with the centers of the ellipses at the same point but the axes of the ellipses are perpendicular to each other so that the long axis of one ellipse is parallel to the short axis of the other ellipse.

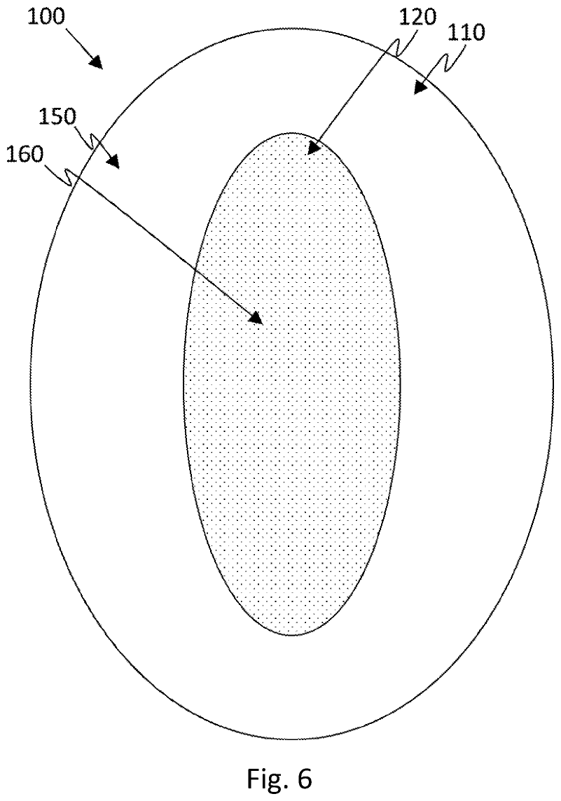

FIG. 6 schematically illustrates a surgical mesh product (100) with an edge region (150) of one elasticity (110) and a central region (160) with a second elasticity (120), where the basic shape of the central region (160) is different from the basic shape of the surgical mesh product (100). In this exemplary embodiment, the basic shape of the surgical mesh product (100) is elliptical, while the central region (160) is basically rectangular.

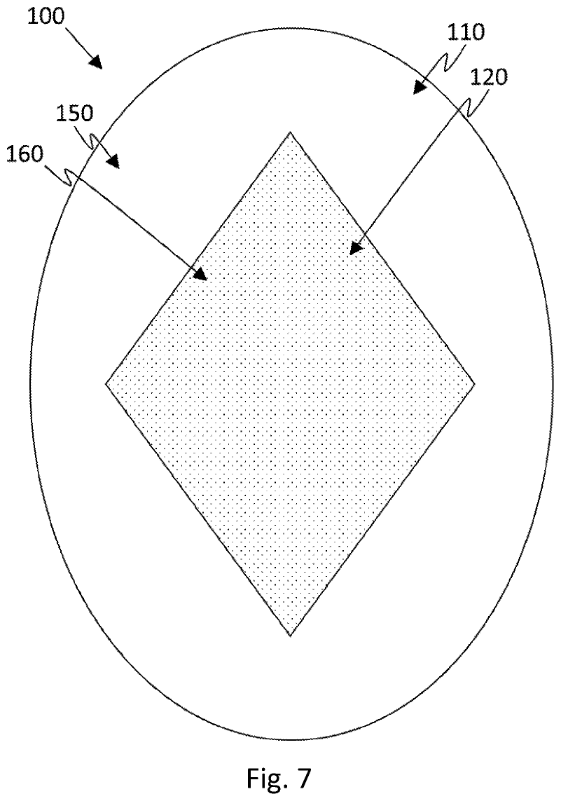

FIG. 7 schematically illustrates a surgical mesh product (100) with an edge region (150) of one elasticity (110) and a central region (160) with a second elasticity (120), where the basic shape of the central region (160) is different from the basic shape of the surgical mesh product (100). In this exemplary embodiment, the basic shape of the surgical mesh product (100) is elliptical, while the central region (160) is diamond-shaped.

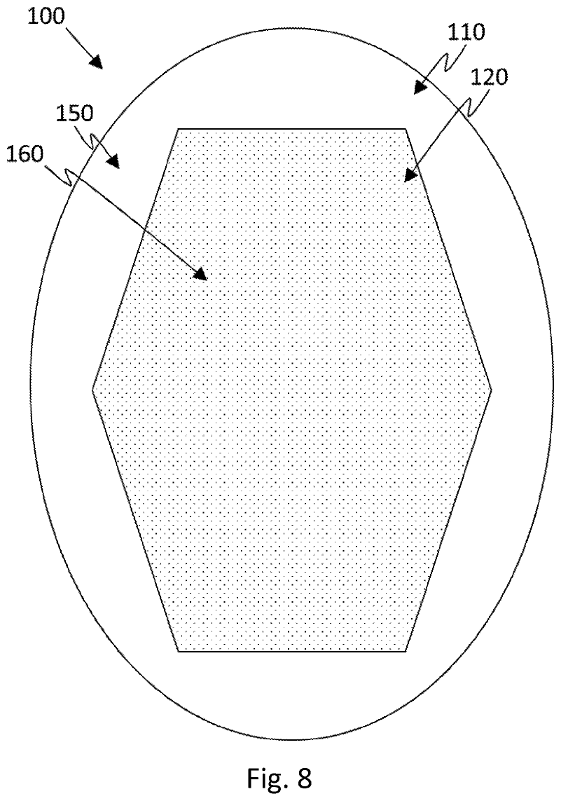

FIG. 8 schematically illustrates a surgical mesh product (100) with an edge region (150) of one elasticity (110) and a central region (160) with a second elasticity (120), where the basic shape of the central region (160) is different from the basic shape of the surgical mesh product (100). In this exemplary embodiment, the basic shape of the surgical mesh product (100) is elliptical, while the central region (160) is basically hexagonal.

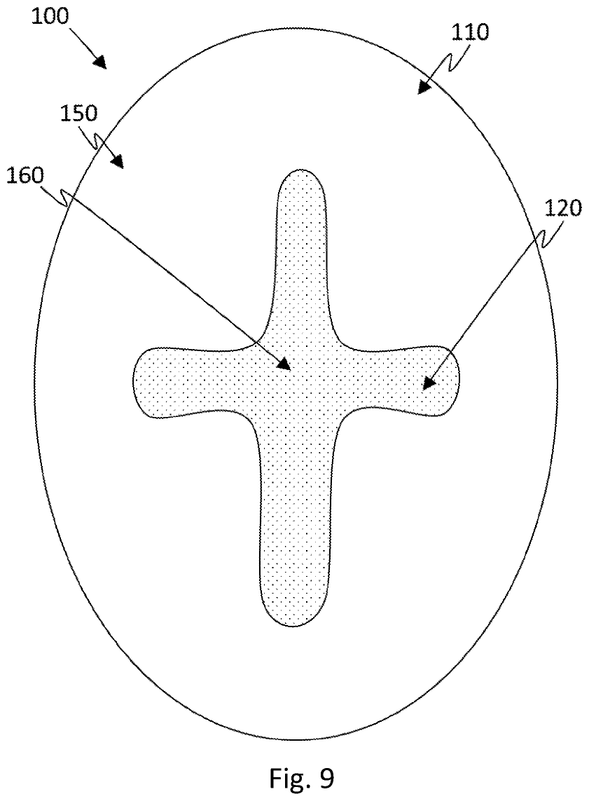

FIG. 9 schematically illustrates a surgical mesh product (100) with an edge region (150) of one elasticity (110) and a central region (160) with a second elasticity (120), where the central region (160) is lobed. In this exemplary embodiment, the central region (160) has four lobes; the number of lobes can range from 3 to 20. In this exemplary embodiment, the lobes and the connections between them are curved. The sides of the lobes can comprise straight sections, curved sections or any combination thereof. The ends of the lobes can comprise straight sections, curved sections or any combination thereof. The connection between adjacent lobes can comprise straight sections, curved sections or any combination thereof.

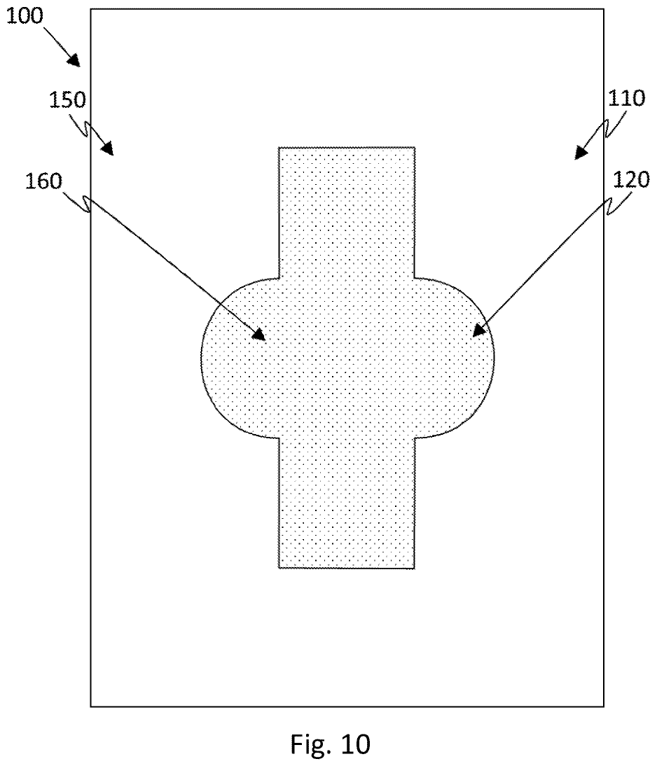

FIG. 10 schematically illustrates a rectangular surgical mesh product (100) with an edge region (150) of one elasticity (110) and a central region (160) with a second elasticity (120), where the central region (160) has four lobes. In this exemplary embodiment, the upper and lower lobes are rectangular while the left and right lobes are circular.

Embodiments can either have or not have left-right symmetry. Embodiments can either have or not have top-bottom symmetry. For a non-limiting (and not very practical) example, a four-lobed central region (not shown) could have two lobes in an upper right quadrant, one lobe in an upper left quadrant, and one lobe pointing downward at an angle to the vertical axis of the surgical mesh product (100) so that most of the downward lobe is in the bottom left quadrant.

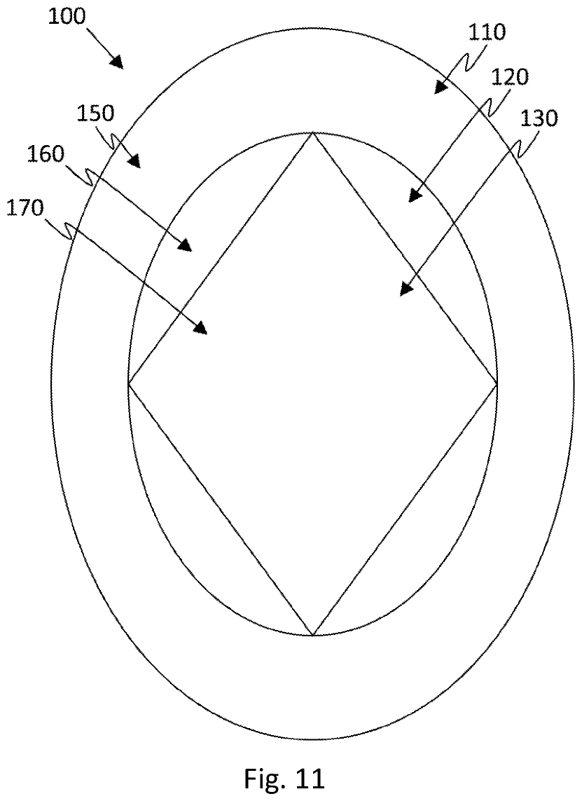

FIG. 11 schematically illustrates a surgical mesh product (100) with an edge region (150) of one elasticity (110) an outer central region (160) with a second elasticity (120) and an inner central region (160) with a third elasticity (120), where none of the basic shapes are the same. In this exemplary embodiment, the basic shape of the surgical mesh product (100) is elliptical, while the outer central region (160) is rectangular and the inner central region (170) is diamond-shaped.

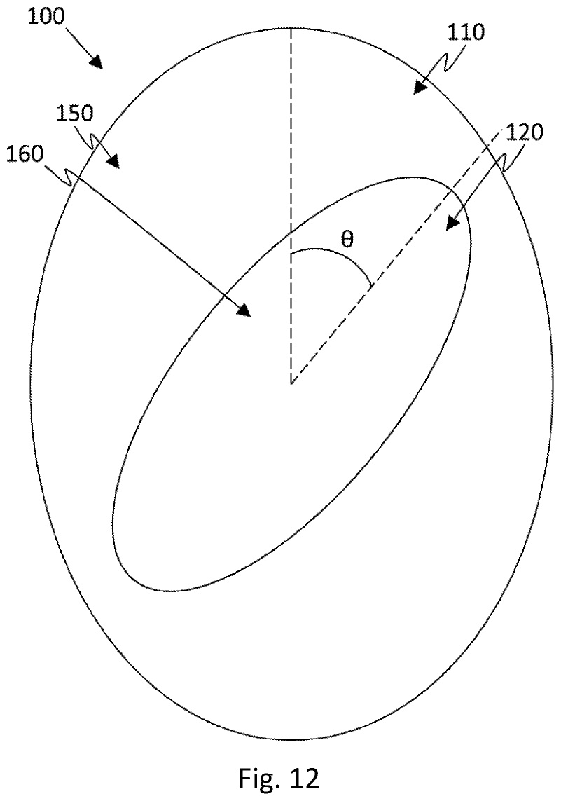

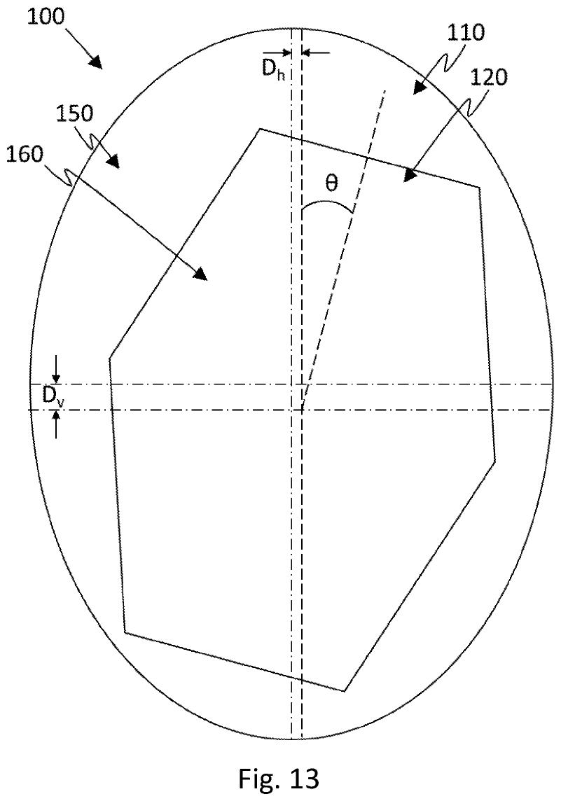

In some embodiment, at least one central regions (160, 170) is not be symmetrically placed within at least one region outer to itself, for example, the surgical mesh product (100) or another central region. As shown in FIGS. 12-13, a central point of a central region (160) can be displaced laterally from a central point of the surgical mesh product (100), a central point of the central region (160) can be displaced vertically from a central point of the surgical mesh product (100), a principal axis of the central region (160) can be rotated relative to a principal axis of the surgical mesh product (100) or any combination thereof. Similarly, a central point of an inner central region (170) can be displaced laterally from a central point of an outer central region (170), a central point of an inner central region (170) can be displaced vertically from a central point of an outer central region (160), a principal axis of an inner central region (170) can be rotated relative to a principal axis of an outer central region (160) or any combination thereof.

FIG. 12 schematically illustrates a surgical mesh product (100) with an edge region (150) of one elasticity (110) and a central region (160) with a second elasticity (120), where the basic shape of the central region (160) is the same as the basic shape of the surgical mesh product (100), in this case elliptical, with the centers of the ellipses at the same point, but the axes of the central ellipse (160) are at an angle θ relative to the axes of the outer ellipse of the surgical mesh product (100). FIG. 13 schematically illustrates a surgical mesh product (100) with an edge region (150) of one elasticity (110) and a central region (160) with a second elasticity (120), where the basic shape of the central region (160) is the same as the basic shape of the surgical mesh product (100), in this case elliptical, with the centers of the ellipses at the same point, but the axes of the central ellipse (160) are at an angle θ relative to the axes of the outer ellipse of the surgical mesh product (100).

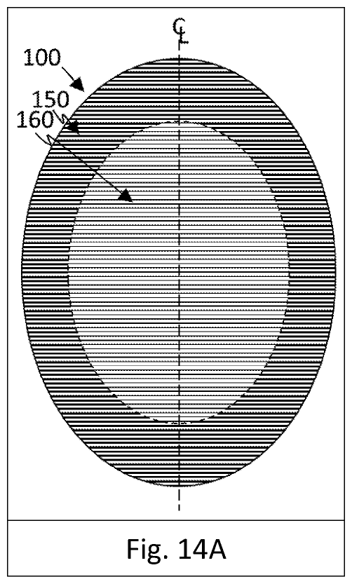

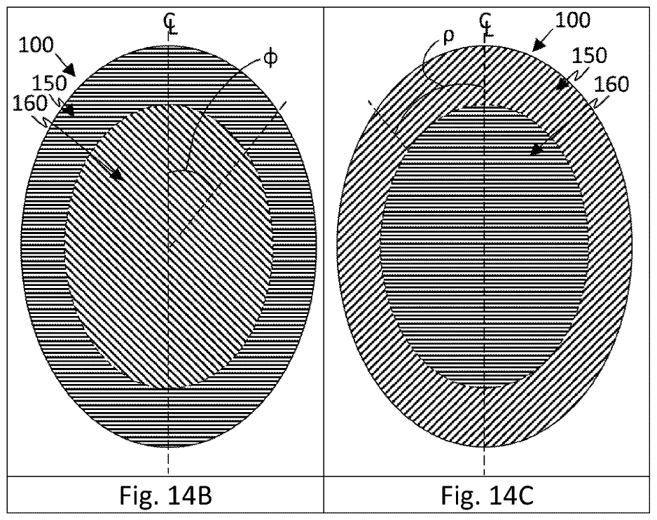

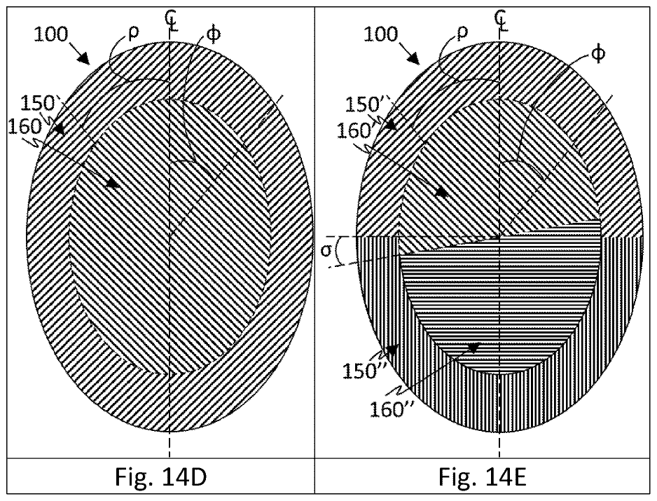

FIG. 14A-E schematically illustrates a surgical mesh product (100) with an edge region (150) of at least one elasticity (110) and a central region (160) with at least one second elasticity (120), where the difference in elasticity is at least partly generated by a difference in direction of the knit, crochet or weave forming the mesh.

In FIG. 14A-E, an exemplary mesh direction is shown by the lines. The mesh direction can be a warp direction or a weft direction. The warp and weft directions can be perpendicular to each other or non-perpendicular to each other. The warp direction can be clearly discerned, the weft direction can be clearly discerned, and any combination thereof.

FIG. 14A schematically illustrates a surgical mesh product (100) with an edge region (150) and a central region (160), where both the mesh direction of the edge region (150) and the mesh direction of the central region (160) are perpendicular to the center line (CL) of the surgical mesh product (100).

FIG. 14B schematically illustrates a surgical mesh product (100) with an edge region (150) and a central region (160), where the mesh direction of the edge region (150) is perpendicular to the center line (CL) of the surgical mesh product (100) and the mesh direction of the central region (160) is at a non-zero angle φ to the center line (CL).

FIG. 14C schematically illustrates a surgical mesh product (100) with an edge region (150) and a central region (160), where the mesh direction of the edge region (150) is at a non-zero angle p to the center line (CL) of the surgical mesh product (100) and the mesh direction of the central region (160) is perpendicular to the center line (CL).

FIG. 14D schematically illustrates a surgical mesh product (100) with an edge region (150) and a central region (160), where the mesh direction of the edge region (150) is at a non-zero angle p to the center line (CL) of the surgical mesh product (100) and the mesh direction of the central region (160) is at a non-zero angle φ to the center line (CL).

FIG. 14E schematically illustrates a surgical mesh product (100) with an edge region comprising two portions (150′, 150″) and a central region comprising two portions (160′, 160″), where the mesh direction of one portion of the edge region (150′) is at a non-zero angle p to the center line (CL) of the surgical mesh product (100), the mesh direction of the second portion of the edge region (150″) is perpendicular to the center line (CL), the mesh direction of one portion of the central region (160′) is at a non-zero angle o to the center line (CL) and the mesh direction of the second portion of the center region (160″) is perpendicular to the center line (CL).

In the exemplary embodiment of FIG. 14E, the boundary between the two portions of the edge region (150′, 150″) is perpendicular to the center line (CL) while the boundary between the two portions of the edge region (160′, 160″) is at a non-zero angle o to the center line (CL).

Boundaries between portions of edge regions or portions of central regions can be sharp (the elasticity changes suddenly), the elasticity can be graded, or any combination thereof.

Boundaries between portions of edge regions or portions of central regions can be straight, curved, or any combination thereof.

The number of central regions can be in a range from 1 to 5.

The elasticity (110) of the edge region (150) can be constant. The elasticity (110) of the edge region (150) can vary radially, axially, or any combination thereof.

The elasticity (120, 130) of at least one central region (160, 170) can be constant. The elasticity (120, 130) of at least one central region (160, 170) can vary radially, axially, or any combination thereof.

A boundary between adjacent regions (150, 160, 170) can be sharp (the elasticity changes suddenly), the elasticity can be graded between adjacent regions (150, 160, 170), or any combination thereof. For non-limiting example, a surgical mesh product has an edge portion with an elasticity of 15% and a central portion with an elasticity of 40%. The boundary between them comprises a graded outer portion and a graded inner portion. In the outer portion of the boundary, the elasticity increases smoothly from 15% elasticity to 20% elasticity. The elasticity then jumps from 20% to 30% and, in the inner portion of the boundary, the elasticity increases from 30% to 40%.

A surgical mesh or a surgical mesh product can comprise any combination of the above features.

In some methods of implanting the novel surgical mesh of the present invention, the fixation devices are sutures, with the sutures fastened using a novel suture device. Embodiments of a novel suture insertion device are disclosed hereinbelow (FIGS. 15-21).

One embodiment of the present invention comprises one or more multiple interconnected ribs, called an umbrella-type suture device. It has a stem that comprises an inner shaft interconnected to the ribs by a hinge and an outer shaft slider interconnected to supports, further connected to the ribs. In this configuration, the outer shaft slider is able to slide up and down the inner shaft and to control the position and opening of the ribs by positioning the support.

1-32. (canceled)

33. A surgical mesh comprising:
a first region having at least one first elasticity; and
at least one second region, each of said at least one second region having at least one second elasticity, said first region surrounding said at least one second region;
wherein, for each suture linking a portion of the first region to tissue, said at least one first elasticity and said at least one second elasticity are configured to move in situ said portion of the first region no more than 1mm in relation to said tissue;
further wherein by means of selection of said at least one first elasticity, said at least one second elasticity, and a member of a group comprising a shape of said first region, a size of said first region, a shape of the at least one second region, a size of the at least one second region, a type of fixation device, a spacing between the fixation devices, or any combination thereof, said surgical mesh is configured to prevent detachment of a surgical mesh product from its fixation devices.

34. The surgical mesh of claim 33, wherein at least one of the following is held:
(i) said at least one second region is entirely contained within said first region; or
(ii) either symmetrically located within said first region or asymmetrically located within said first region.

35. The surgical mesh of claim 34, wherein said asymmetrical location is provided by a member of a group consisting of a location of a center of said at least one second region is different from a location of a center of said first region, an axis of said at least one second region is rotated relative to an axis of said first region or any combination thereof.

36. The surgical mesh of claim 33, wherein said at least one second region comprises a plurality of second regions.

37. The surgical mesh of claim 36, wherein, for at least one pair from said plurality of second regions, said second regions have different elasticities.

38. The surgical mesh of claim 36, wherein at least of the following is held true:
(i) at least one of said plurality of second regions is entirely contained within at least one other of said plurality of second regions; or
(ii) either symmetrically located within another of said plurality of second regions or asymmetrically located within said another of said plurality of second regions.

39. The surgical mesh of claim 38, wherein said asymmetrical location is provided by a member of a group consisting of a location of a center of said at least one second region is different from a location of a center of said first region, an axis of said at least one second region is rotated relative to an axis of said first region or any combination thereof.

40. The surgical mesh of claim 33, wherein said first region differs from said at least one second region by a difference selected from a group consisting of filament type, filament material, filament diameter, filament shape, mesh type, pore diameter, thickness or any combination thereof.

41. The surgical mesh of claim 40, wherein at least one of the following is held true:
(i) said filament type is selected from a group consisting of monofilament, multifilament or any combination thereof;
(ii) said filament material is selected from a group consisting of metal, a composite, a polymer, a biodegradable biomaterial or any combination thereof; or
(iii) said mesh type is selected from a group consisting of woven, knitted, crocheted, non-woven or any combination thereof.

42. The surgical mesh of claim 33, wherein a shape of said at least one second region is selected from a group consisting of elliptical, polygonal, oval, lobed or any combination thereof.

43. The surgical mesh of claim 42, wherein said shape of said at least one second region is either the same as a shape of said at least one first region, or different from a shape of said at least one first region.

44. The surgical mesh of claim 42, wherein a shape of one of said plurality of second regions is either the same as a shape of another of said plurality of second regions, or different from a shape of another of said plurality of second regions.

45. A method of attaching a surgical mesh comprising steps of:
providing a surgical mesh comprising:
a first region having at least one first elasticity; and
at least one second region, each of said at least one second region having at least one second elasticity, said first region surrounding said at least one second region;
placing edges of a cut region in apposition;
emplacing the surgical mesh such that external edges of the surgical mesh extend beyond all boundaries of said cut region;
suturing the surgical mesh to an underlying material, said underlying material being flexible;
wherein, for each suture linking a portion of the first region to said underlying material, said at least one first elasticity and said at least one second elasticity are configured, when said underlying material is moved or stretched, to move said portion of the first region no more than 1 mm in relation to said underlying material;
further wherein by means of selection of said at least one first elasticity, said at least one second elasticity, and a member of a group comprising a shape of said first region, a size of said first region, a shape of the at least one second region, a size of the at least one second region, a type of fixation device, a spacing between the fixation devices, or any combination thereof, said surgical mesh is configured to prevent detachment of a surgical mesh product from its fixation devices.

46. The method of claim 45, additionally comprising a step of
(i) providing said at least one second region entirely contained within said first region;
(ii) providing said at least one second region symmetrically located within said first region or asymmetrically located within said first region; or
(iii) both

47. The method of claim 46, additionally comprising a step of providing said asymmetrical location by a member of a group consisting of a location of a center of said at least one second region is different from a location of a center of said first region, an axis of said at least one second region is rotated relative to an axis of said first region or any combination thereof.

48. The method of claim 46, wherein said at least one second region comprising a plurality of second regions.

49. The method of claim 48, additionally comprising a step of
(i) providing said second regions having different elasticities, for at least one pair from said plurality of second regions;
(ii) providing at least one of said plurality of second regions entirely contained within at least one other of said plurality of second regions;
(iii) providing said at least one of said plurality of second regions either symmetrically located within another of said plurality of second regions or asymmetrically located within said another of said plurality of second regions;
(iv) providing said first region differing from said at least one second region by a difference selected from a group consisting of filament type, filament material, filament diameter, filament shape, mesh type, pore diameter, thickness or any combination thereof;
(v) any combination thereof.

50. The method of claim 49, additionally comprising a step of
(i) providing said asymmetrical location by a member of a group consisting of a location of a center of said at least one second region is different from a location of a center of said first region, an axis of said at least one second region is rotated relative to an axis of said first region or any combination thereof;
(ii) selecting said filament type from a group consisting of monofilament, multifilament or any combination thereof;
(iii) selecting said filament material from a group consisting of metal, a composite, a polymer, a biodegradable biomaterial or any combination thereof;
(iv) selecting said mesh type from a group consisting of woven, knitted, crocheted, non-woven or any combination thereof; or
(v) any combination thereof.

51. The method of claim 48, additionally comprising a step of selecting a shape of said at least one second region from a group consisting of elliptical, polygonal, oval, lobed or any combination thereof.

52. The method of claim 51, additionally comprising
(i) a step of providing said shape of said at least one second region either the same as a shape of said at least one first region, or different from a shape of said at least one first region;
(ii) providing a shape of one of said plurality of second regions either the same as a shape of another of said plurality of second regions, or different from a shape of another of said plurality of second regions; or
(iii) both.
